(19) United States
(12) Patent Application Publication
(10) Pub. No.: US 2019/0295688 A1
(43) Pub. Date: Sep. 26, 2019

(54) PROCESSING BIOLOGICAL SEQUENCES USING NEURAL NETWORKS

(71) Applicant: Google LLC, Mountain View, CA (US)

(72) Inventors: Mark Andrew DePristo, Los Altos, CA (US); Akosua Pokua Busia, Sunnyvale, CA (US); George Edward Dahl, Sunnyvale, CA (US)

(21) Appl. No.: 16/364,095

(22) Filed: Mar. 25, 2019

Related U.S. Application Data
(60) Provisional application No. 62/647,580, filed on Mar. 23, 2018.

(51) Int. Cl. G16B 30/00; G06N 3/08; G06N 3/04

(52) U.S. Cl. CPC G16B 30/00; G06N 3/04; G06N 3/08

(57) ABSTRACT

Methods, systems, and apparatus, including computer programs encoded on computer storage media, for processing a biological sequence using a neural network. One of the methods includes obtaining data identifying a biological sequence; generating, from the obtained data, an encoding of the biological sequence; processing the encoding using a deep neural network, wherein the deep neural network is configured through training to process the encoding to generate a score distribution over a set of biological labels for the biological sequence; and classifying the biological sequence using the score distribution.

### CROSS-REFERENCE TO RELATED APPLICATION

This application claims priority to U.S. Provisional Application No. 62/647,580, filed on Mar. 23, 2018. The disclosure of the prior application is considered part of and is incorporated by reference in the disclosure of this application.

### BACKGROUND

This specification relates to neural networks for processing biological sequences. Neural networks are machine learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters.

### SUMMARY

This specification describes a system implemented as computer programs on one or more computers in one or more locations that processes data representing a biological sequence using a deep neural network to generate a score distribution over biologically meaningful labels, e.g., database labels, for the biological sequence. For example, the biological sequence can be an RNA sequence and the labels can be taxonomical labels, e.g., labels at the superkingdom, phylum, class, order, family, genus, or species levels.

Particular embodiments of the subject matter described in this specification can be implemented so as to realize one or more of the following advantages.

By using a deep neural network that is trained directly on encoded sequence—label pairs as described in this specification, the described systems can assign labels to sequences more accurately than existing approaches, e.g., existing alignment based approaches. Additionally, the described systems can make predictions effectively even when the input data is noisy, i.e., the deep neural network is robust to noisy or ambiguous sequence data. In particular, to make the deep neural network more robust and allow the deep neural network to effectively be employed when the input data is noisy or includes ambiguous sequence data, noise can be injected into the training process as described in this specification.

In particular, the biological sequences can be short RNA or DNA sequences, i.e., short sequencing reads that include a small number of base pairs, i.e., 200 base pairs or less. In particular, the biological sequences can be sequencing reads that include 25, 50, 100, 150, or 200 base pairs. Conventional techniques have difficulties accurately classifying such short sequences, particularly when inputs can be noisy or highly ambiguous (i.e., can include large numbers of ambiguity codes). By making use of a deep neural network as described in this specification, however, the described systems are able to classify sequences of this length with a high degree of accuracy, even when operating on noisy and/or ambiguous inputs.

Additionally, the deep neural network that operates on the encoded sequence includes multiple depthwise separable convolutional layers followed by multiple fully-connected layers. Making use of depthwise separable convolutions allows the deep neural network to remain computationally efficient while making high-quality predictions. In particular, the architecture of the neural network leverages the translational equivariance of the biological sequences. To allow the neural network to operate on variable length sequence reads, i.e., sequences of variable size, the fully-connected layers can be tiled and can be followed by a pooling layer, e.g., an average pooling layer, that pools the outputs of the fully-connected layers before they are processed by a softmax output layer to generate the score distribution over the labels. Thus, the neural network can be used to process sequence reads of different lengths than the sequence reads that the neural network was trained on.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example neural network system.

FIG. 2 is a diagram illustrating an example architecture of the deep neural network.

FIG. 3 is a flow diagram of an example process for processing a biological sequence using the deep neural network.

FIG. 4 is a diagram illustrating an example encoding of example biological sequence data.

FIG. 5 is a flow diagram of an example process for training the deep neural network.

Like reference numbers and designations in the various drawings indicate like elements.

### DETAILED DESCRIPTION

This specification describes a system implemented as computer programs on one or more computers in one or more locations that processes data representing a biological sequence using a deep neural network to generate a score distribution over biologically meaningful labels, e.g., database labels, for the biological sequence. For example, the biological sequence can be an RNA sequence and the labels can be taxonomical labels, e.g., labels at the superkingdom, phylum, class, order, family, genus, or species levels.

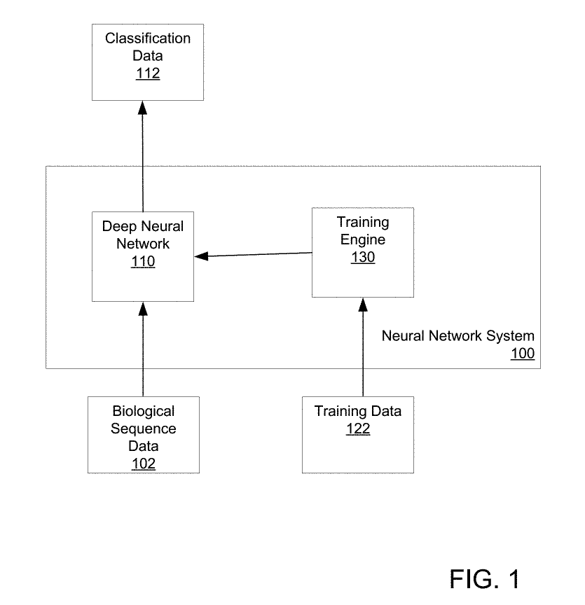

FIG. 1 shows an example neural network system 100. The neural network system 100 is an example of a system implemented as computer programs on one or more computers in one or more locations, in which the systems, components, and techniques described below can be implemented.

The neural network system 100 processes data representing a biological sequence 102 using a deep neural network 110 to generate a score distribution over biologically meaningful labels, e.g., database labels, for the biological sequence 102. The system 100 then classifies the biological sequence 102 using the score distribution, i.e., generates and outputs a classification 112 using the score distribution. The classification 112 can identify one or more highest-scoring labels for the biological sequence or can, as described below, represent other data derived from the score distribution.

Once generated, the system 100 can store the classification 112 in association with the data representing the sequence 102 or can provide the classification 112 to another system for further processing or for presentation to a user on a user device. Alternatively or in addition, the system 100 can output the score distribution for the biological sequence 102 as generated by the deep neural network 110.

The system 100 can process data representing any of a variety of biological sequences to generate any a variety of biologically meaningful predictions for the biological sequence.

For example, the biological sequence can be an RNA (ribonucleic acid) sequence or a DNA (Deoxyribonucleic acid) sequence and the labels can be taxonomical labels, e.g., labels at the superkingdom, phylum, class, order, family, genus, or species levels. As another example, the biological labels can be a set of operational taxonomic units for the RNA or DNA. As yet another example, the biological labels can be a set of gene labels or a set of gene property labels. As yet another example, the biological labels can be labels that identify a pathogenicity of the biological sequence.

In particular, the biological sequences can be short RNA or DNA sequences, i.e., short sequencing reads that include a small number of base pairs, i.e., 200 base pairs or less. In particular, the biological sequences can be sequencing reads that include 25, 50, 100, 150, or 200 base pairs. Conventional techniques have difficulties accurately classifying such short sequences, particularly when inputs can be noisy or highly ambiguous (i.e., can include large numbers of ambiguity codes). By making use of a deep neural network as described in this specification, however, the described systems are able to classify sequences of this length with a high degree of accuracy, even when operating on noisy and/or ambiguous inputs.

As another example, the biological sequence can be a protein sequence and the set of biological labels can be a set of possible protein functions for the protein.

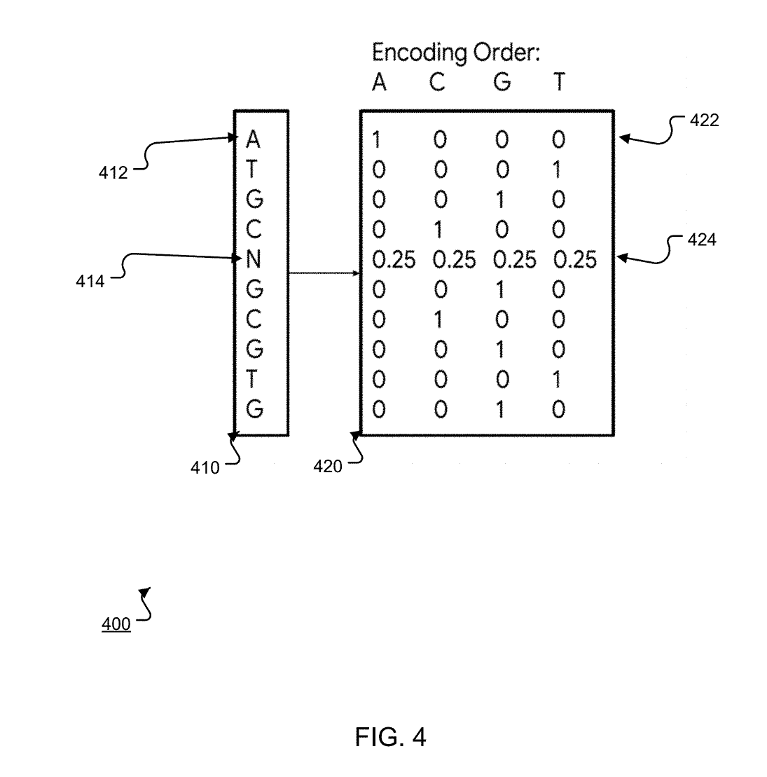

Generally, the data representing the biological sequence is a sequence of canonical compounds and ambiguity codes that collectively represent the biological sequence. For example, when the biological sequence is RNA, the data can be a sequence of canonical nitrogenous bases and IUPAC ambiguity codes. An example representation of RNA will be described in more detail below with reference to FIG. 4.

Generally, the deep neural network 110 is configured to receive an encoding of a biological sequence and to process the encoding to generate the score distribution over a set of possible biological labels for the biological sequence. The encoding is a representation of the biological sequence in a form that can be efficiently processed by the neural network 110. Generating an encoding of a biological sequence will be described below with reference to FIG. 4.

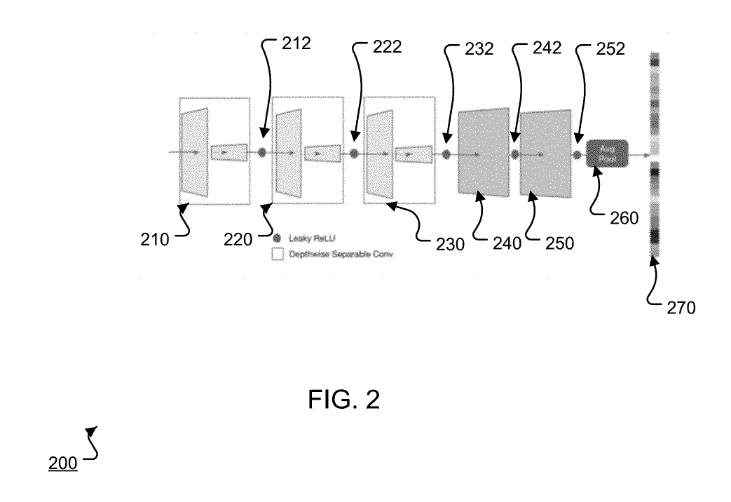

To leverage the equivariance of the biological sequences and to remain computationally efficient while making high-quality predictions, the deep neural network 110 employs depthwise separable convolutional layers, which are more computationally efficient than conventional convolutional layers. The architecture of the deep neural network 110 will be described in more detail below with reference to FIG. 2.

In order to allow the deep neural network 110 to generate accurate score distributions, i.e., that accurately reflect the actual labels for input biological sequences, the system 100 includes a training engine 130 that trains the neural network 110 on training data 122. In particular, the training data 122 includes data representing a plurality of biological sequences and respective biological labels for each of the biological sequences. The training engine 130 trains the deep neural network on the training data 122 using supervised learning to update the values of the parameters of the neural network 110 so that the neural network 110 generates score distributions that accurately reflect the biological labels for the biological sequences in the training data 122.

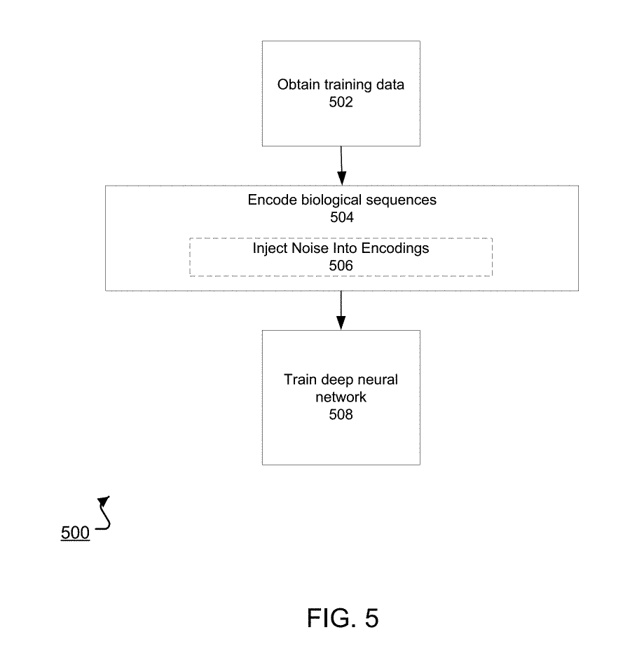

Training the deep neural network 110 will be described in more detail below with reference to FIG. 5.

FIG. 2 is a diagram 200 illustrating an example architecture of the deep neural network 110.

Generally, as described above, the deep neural network 110 is configured to receive an encoding of a biological sequence and to process the encoding to generate a score distribution over a set of possible biological labels for the biological sequence.

In particular, in the example of FIG. 2, the deep neural network 110 includes multiple depthwise separable convolutional layers 210, 220, and 230 followed by multiple fully-connected layers 240 and 250.

In cases where the data identifying the biological sequence is longer than the length of data that the neural network 110 is configured to process, the fully-connected layers can be tiled and the tiled outputs from the last fully-connected layer 250 for multiple segments of the biological sequence can be processed through a pooling layer 260, e.g., an average pooling layer, before the pooled outputs are processed by a softmax layer 270 that is configured to generate a score distribution, e.g., a probability distribution, over the possible labels for the biological sequence. In other words, the system can split the encoding of the biological sequence into tiles and process each tile through the layers 210-250. The system can then tile the outputs of the last fully-connected layer 250 and process the tiled output through the average pooling layer 260 to generate the input to the softmax layer 270.

In cases where the data identifying the biological sequence is not longer than the length of data that the neural network 110 is configured to process, the pooling layer 260 is not needed and the softmax output layer 270 can directly process the output generated by the last fully-connected layer 250.

In some cases, the system maintains multiple different instances of the neural network 110, each of which has been configured and trained to process encodings of different size, i.e., to process sequence reads of different lengths.

Each of the layers 210 through 250 applies a linear transformation to the input to the layer and then applies an element-wise non-linear activation function 212, 222, 232, 242, or 252 to the output of the linear transformation to generate the final output of the layer. For some or all of the layers, the activation function can be a leaky rectified-linear unit activation (reLU) function. A leaky reLU function is a function that outputs, given an input element x, the maximum of x and ax, where a is a fixed slope value that is between zero and one, exclusive. Thus, when x is greater than or equal to zero, the leaky reLU outputs x and when x is less than zero, the leaky reLU outputs ax.

For the depthwise separable convolutional layers 210-230, the linear transformation applied by the layer is a depthwise separable convolution. A depthwise separable convolution is performed by first performing a spatial convolution applied independently over each channel of the input followed by a pointwise convolution across channels. By employing depthwise separable convolutions to process the encoding, e.g., in place of conventional convolutional layers, the neural network 110 can more efficiently make use of the available computational resources in order to generate accurate predictions, i.e., because depthwise separable convolutions make use of parameters more efficiently than conventional convolutional layers.

For the fully-connected layers 240 and 250, the linear transformation applied by the layer is a matrix multiplication between a parameter matrix for the layer and the input to the layer, optionally followed by an addition of a bias.

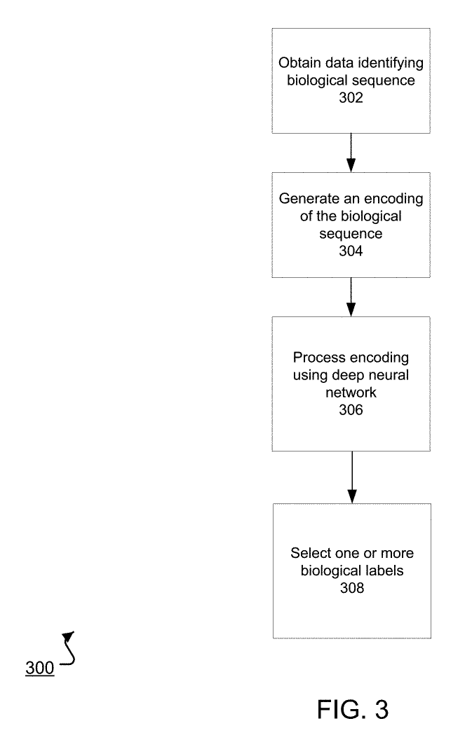

FIG. 3 is a flow diagram of an example process 300 for processing a biological sequence using a deep neural network. For convenience, the process 300 will be described as being performed by a system of one or more computers located in one or more locations. For example, a neural network system, e.g., the neural network system 100 of FIG. 1, appropriately programmed, can perform the process 300.

The system receives data identifying a biological sequence (step 302). As described above, the data is a sequence of canonical compounds and ambiguity codes that collectively represent the biological sequence.

The system generates, from the obtained data, an encoding of the biological sequence (step 304). Generally, the encoding is a two-dimensional representation of the obtained data that can effectively be processed by the deep neural network, i.e., by the input depthwise separable convolutional layer of the neural network. An example technique for generating the encoding will be described below with reference to FIG. 4.

The system processes the encoding using the deep neural network to generate a score distribution over a set of biological labels for the biological sequence (step 306).

In some cases, the encoding is not larger than the size that the deep neural network is configured to process. In these implementations, the system can process the encoding in a single pass through the deep neural network to generate the score distribution and does not need to employ a deep neural network with an average pooling layer.

In some cases, the encoding is larger than the size that the deep neural network is configured to process. In these implementations, the system can divide the encoding into tiles, each of which has a size that is equal to the size that the deep neural network is configured to process. The system can then generate the encoding by tiling the fully-connected layers. That is, the system can process each tile through the convolutional layers and the fully-connected layers, tile the outputs generated by the last fully-connected layer, and process the tiled outputs through the average pooling layer. The system can then process the pooled output through the softmax output layer to generate the score distribution. The system classifies the biological sequence using the score distribution (step 308).

In some implementations, the system classifies the biological sequence by selecting one or more highest-scoring labels according to the score distribution and providing the selected labels and, optionally, the corresponding scores from the distribution as the classification of the biological sequence.

In some other implementations, the required classification is at a higher level of the taxonomy than the score distribution generated by the neural network. For example, the required classification may be to classify the order or the genus to which the biological sequence belong, while the score distribution is a probability over a set of possible species. In these implementations, to compute a new probability distribution over the required higher taxonomic labels, i.e., the labels at the specified higher taxonomic level, the system marginalizes the species-level distribution produced by the neural network by summing the probability assigned to all species under each taxon, i.e., to all species under each order or genus, in the species-level distribution. The system can then classify the biological sequence by selecting one or more highest-scoring labels according to the higher-level distribution and providing the selected labels and, optionally, the corresponding scores from the higher-level distribution as the classification of the biological sequence as the classification of the sequence.

FIG. 4 is a diagram 400 illustrating an example encoding 420 of example biological sequence data 410.

In particular, the biological sequence data 410 is a sequence of canonical compounds and ambiguity codes that collectively represent the biological sequence. In the example of FIG. 4, the biological sequence is RNA and each element in the sequence either represents a canonical nitrogenous bases (adenine (A), cytosine (C), guanine (G), thymine (T)) or is an IUPAC ambiguity code (K, M, R, Y, S, W, B, V, H, D, X, N). Each ambiguity code represents that the sequence element can be any one of a corresponding subset of canonical compounds. For example, the code N represents that the sequence element can be any one of A or C or T or G while the code M represents that the sequence element can only be either A or C.

To generate the encoding 420 from the sequence data 410, the system generates a two-dimensional representation, e.g., a matrix that has a respective row for each sequence element. In particular, the system one-hot encodes each of the canonical compounds and resolves each ambiguity code to a corresponding probability distribution over the canonical compounds. In other words, for each sequence element that represents a canonical compound, the system includes a one-hot vector that represents the canonical compound in the encoding 420. For example, for the element 412 (“A”), the system includes a one-hot vector 422, i.e., (1, 0, 0, 0), that represents the canonical base A. For each ambiguity code, the system generates a vector that reflects, for each canonical compound, the probability that the corresponding sequence element is the canonical compound. For example, for the sequence element 414 (“N”), the system generates a vector 424 that represents that the element is equally likely to be any of the compounds A, C, G, or T, i.e., (0.25, 0.25, 0.25, 0.25).

FIG. 5 is a flow diagram of an example process for training the deep neural network. For convenience, the process 500 will be described as being performed by a system of one or more computers located in one or more locations. For example, a neural network system, e.g., the neural network system 100 of FIG. 1, appropriately programmed, can perform the process 500.

The system obtains training data for the deep neural network (step 502). The training data includes data representing a plurality of biological sequences and respective biological labels for each of the biological sequences, i.e., a respective biological label that identifies one or more labels from the set of possible labels that apply to the biological sequence.

The system generates an encoding for each of the biological sequences (step 504), i.e., as described above with reference to FIG. 4.

Optionally, as part of generating the encodings, the system randomly injects noise into the encodings for the biological sequence (step 506). Injecting noise into the encodings can help make the trained deep neural network robust to noise and ambiguity in data that is received after training.

To inject noise in the encodings, the system determines whether to modify each element in at least at least a subset of the biological sequences. In particular, for each element of a given biological sequence that is in the subset, the system determines to modify the element with a fixed probability r (where r is a fixed value between zero and one, exclusive). If the system determines to modify the element and the element is a canonical compound, the system flips, i.e., modifies or changes, the canonical compound to one of the other canonical compounds with equal probability. If the system determines to modify the element and the element is an ambiguity code, the system flips the element to one of the canonical compounds with equal probability. These random variations in the biological sequences have the effect of injecting noise that increases the robustness of the trained model.

The system then generates the encoding of the (potentially modified) biological sequences as described above. In cases where the system randomly injected noise as described above, the encodings reflect the potentially noisy modified sequences rather than the originally received sequences.

The system trains the deep neural network on the training data using supervised learning (step 508) to cause the neural network to generate score distributions that accurately reflect the biological labels for the biological sequences in the training data. In particular, the system can repeatedly process encodings of biological sequences in the training data and update the parameters of the deep neural network, i.e., the parameters of the convolutional layers, the fully-connected layers, and the softmax layer, to minimize a cross-entropy loss that measures an error between (i) the score distribution generated by the neural network by processing the encoding of a biological sequence and (ii) the biological label for the biological sequence in the training data. The system can use any appropriate supervised learning technique to perform the parameter updates. For example, the system can use gradient descent with the Adam optimizer (optionally, with gradient clipping), the RMSprop optimizer, or the SGD optimizer.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more computers, the method comprising:
obtaining data identifying a biological sequence;
generating, from the obtained data, an encoding of the biological sequence;
processing the encoding using a deep neural network, wherein the deep neural network is a convolutional neural network that comprises a plurality of depthwise separable convolutional layers that operate on the encoding of the biological sequence, and wherein the deep neural network has been configured through training to process the encoding to generate a score distribution over a set of biological labels for the biological sequence; and
classifying the biological sequence using the score distribution.

2. The method of claim 1, wherein the biological sequence is RNA.

3. The method of claim 1, wherein the biological sequence is DNA.

4. The method of claim 1, wherein the set of biological labels are a set of taxonomic labels for the biological sequence.

5. The method of claim 4, wherein the set of taxonomic labels comprises a set of species labels for the biological sequence.

6. The method of claim 1, wherein the biological labels are a set of operational taxonomic units.

7. The method of claim 1, wherein the biological labels are a set of gene labels or a set of gene property labels.

8. The method of claim 1, wherein the biological labels comprise labels that identify a pathogenicity of the biological sequence.

9. The method of claim 1, wherein the biological sequence is a protein.

10. The method of claim 9, wherein the set of biological labels are a set of possible protein functions for the protein.

11. The method of claim 1, wherein the sequence is a sequence of canonical compounds and ambiguity codes, and wherein generating the encoding for the sequence comprises:
one-hot encoding each of the canonical compounds; and
resolving each ambiguity code to a corresponding probability distribution over the canonical compounds.

12. The method of claim 11, further comprising:
obtaining training data for the deep neural network, the training data comprising:
data representing a plurality of biological sequences and respective biological labels for each of the biological sequences; and
training the deep neural network on the training data using supervised learning to generate score distributions that accurately reflect the biological labels for the biological sequences in the training data.

13. The method of claim 12, further comprising:
wherein training the deep neural network comprises:
randomly injecting noise when encoding the biological sequences in the training data for input to the deep neural network.

14. The method of claim 13, wherein randomly injecting noise comprises:
for each element of a given biological sequence, determining to modify the element with a fixed probability r.

15. The method of claim 14, wherein, when the element is a canonical compound and in response to determining to modify the element, flipping the canonical compound to one of the other canonical compounds with equal probability.

16. The method of claim 14, wherein, when the element is not a canonical compound and in response to determining to modify the element, flipping the element to one of the canonical compounds with equal probability.

17. The method of claim 1, wherein the plurality of depthwise separable convolutional layers are followed by a plurality of fully-connected layers.

18. The method of claim 17, wherein the fully-connected layers are tiled, and wherein the deep neural network comprises a pooling layer following the fully-connected layers and a softmax output layer to generate the score distribution over the labels following the pooling layer.

19. The method of claim 18, wherein the pooling layer is an average pooling layer.

20. The method of claim 19, wherein the deep neural network comprises a softmax output layer to generate the score distribution over the labels following the fully-connected layers.

21. The method of claim 17, wherein the depthwise separable convolutional layers, the plurality of fully-connected layers, or both have a leaky rectified-linear unit activation function.

22. A system comprising one or more computers and one or more storage devices storing instructions that, when executed by the one or more computers, cause the one or more computers to perform operations comprising:
obtaining data identifying a biological sequence;
generating, from the obtained data, an encoding of the biological sequence;
processing the encoding using a deep neural network, wherein the deep neural network is a convolutional neural network that comprises a plurality of depthwise separable convolutional layers that operate on the encoding of the biological sequence, and wherein the deep neural network has been configured through training to process the encoding to generate a score distribution over a set of biological labels for the biological sequence; and
classifying the biological sequence using the score distribution.

23. One or more non-transitory computer-readable storage media storing instructions that, when executed by one or more computers, cause the one or more computers to perform operations comprising:
obtaining data identifying a biological sequence;
generating, from the obtained data, an encoding of the biological sequence;
processing the encoding using a deep neural network, wherein the deep neural network is a convolutional neural network that comprises a plurality of depthwise separable convolutional layers that operate on the encoding of the biological sequence, and wherein the deep neural network has been configured through training to process the encoding to generate a score distribution over a set of biological labels for the biological sequence; and
classifying the biological sequence using the score distribution.
